(19) United States
(12) Patent Application Publication
(10) Pub. No.: US 2015/0282979 A1
(43) Pub. Date: Oct. 8, 2015

(54) FOOT SLEEVE

(71) Applicant: Cherie Christenson, North Bellmore, NY (US)

(72) Inventor: Cherie Christenson, North Bellmore, NY (US)

(21) Appl. No.: 14/247,707

(22) Filed: Apr. 8, 2014

(51) Int. Cl. A61F 7/02; A61G 7/057; A47G 9/02

(52) U.S. Cl. CPC A61F 7/02; A47G 9/02; A61G 7/057; A61F 2007/0045

(57) ABSTRACT

A foot sleeve for use with a bed comprising: a sleeve opening; a top inner surface within the sleeve opening; a bottom inner surface within the sleeve opening; and weighted corners at a distal end of the foot sleeve. In the one particular embodiment, the top inner surface may be covered with fleece material and the bottom inner surface is covered with sheepskin.

### BACKGROUND OF THE INVENTION

1. Field of Invention

The present invention relates to a foot sleeve used to place over a bed sheet.

2. Description of Related Art

Many individuals who bed ridden due to illnesses or disease may have issues regarding circulation to their feet. The lack of circulation to the lower extremities may cause several problems to the patient. Many times the feet may become cold at night and particularly while in bed. Further, pressure ulcers may develop at the lower extremities due to the lack of circulation. In particular patients that may be hospitalized and recovering from illnesses, surgeries or other injuries are exposed to the normally cool environment of a hospital. Many patients complain of cold feet and thus require extra blankets or socks in order to keep their feet warm while hospitalized. One drawback to using socks is that they create moisture and must be changed frequently. Further, the use of excessive blankets can also be cumbersome for both the patient and the facility. Therefore it would be advantageous to have a device that may be used by a patient who is bed-ridden to insert their feet to maintain the warmth of their feet while in the hospital.

### SUMMARY OF THE INVENTION

The present invention relates to a foot sleeve for use with a bed comprising: a sleeve opening; a top inner surface within the sleeve opening; a bottom inner surface within the sleeve opening; and weighted corners at a distal end of the foot sleeve. In the one particular embodiment, the top inner surface may be covered with fleece material and the bottom inner surface is covered with sheepskin.

### BRIEF DESCRIPTION OF DRAWINGS

FIG. 1 depicts a foot sleeve in accordance with the present invention.

FIG. 2A depicts the foot sleeve placed over a mattress in accordance with the present invention.

FIG. 2B shows a side view of the foot sleeve for use over a mattress.

### DETAILED DESCRIPTION

The present invention relates to a foot sleeve to be used by a patient or other bed-ridden individual. The foot sleeve includes an opening that allows the user to place their feet within the sleeve opening as desired. The foot sleeve is placed at the foot of the mattress and allows the patient to insert their feet into and out of the opening as desired. Use of the foot sleeve eliminates the necessity for extra blankets and socks while a person is in the bed.

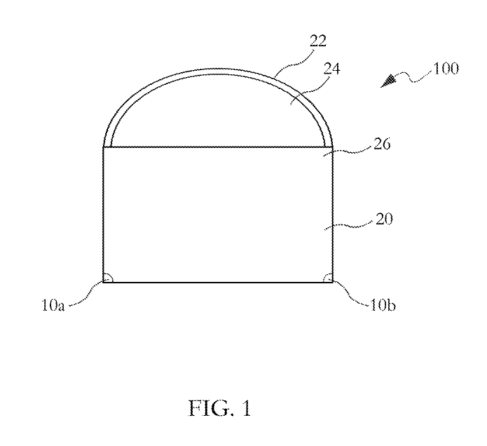

In one particular embodiment, the foot sleeve includes an inner surface that provides a warm and soft texture for placement of the user's feet in the sleeve. In reference to FIG. 1, a foot sleeve 100 in accordance with the present invention is depicted. The foot sleeve 100 includes a sleeve opening 24 that allows for the insertion of a user's feet within the sleeve. The sleeve 100 extends and includes a body portion 20 with weights at the distal end of the sleeve 100. Weights 10a, 10b are provided at each opposing corner of the foot sleeve 100. The weights 10a, 10b will enable the user to place the sleeve in a stationary position at the foot of the mattress or bed. The weights 10a, 10b also help to stabilize the sleeve during use and avoid movement of the sleeve unless so desired by the user.

The foot sleeve 100 includes a top inner surface 26 and a bottom inner surface 22. In one particular embodiment, the bottom inner surface 22 is covered with a sheepskin material. The top inner surface 26 may be covered with a fleece providing a warm, comfortable material within the sleeve 100 for use by the user.

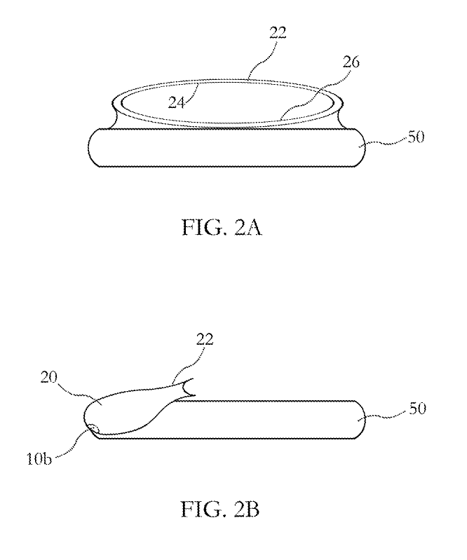

As stated above, the sleeve according to the present invention preferably may placed over a mattress at the foot of the bed which is more clearly shown in FIGS. 2A and 2B. The sleeve 100 is shown over a mattress 50. Preferably the sleeve 100 is placed over a fitted sheet that is normally used with a mattress. The opening 24 is expanded showing the top inner surface 26 and bottom inner surface 22. The foot sleeve according to the present invention therefore creates a suitable environment for the user to place their feet while in the bed. Although particularly designed for a hospital use or other healthcare facility, the present invention may easily be placed in a conventional bed for use in an individual's home.

1. A foot sleeve for use with a bed comprising:
a. a sleeve opening;
b. a top inner surface within the sleeve opening;
c. a bottom inner surface within the sleeve opening;
d. a sleeve length, where the sleeve length covers a foot portion of a mattress;
e. a sleeve width, where the sleeve width extends over each opposing edge of the mattress; and
f. weighted corners at a distal end of the foot sleeve.

2. The foot sleeve according to claim 1, where the top inner surface is covered with fleece material.

3. The foot sleeve according to claim 1, where the bottom inner surface is covered with sheepskin.
